(19) United States
(12) Patent Application Publication
(10) Pub. No.: US 2024/0423863 A1
(43) Pub. Date: Dec. 26, 2024

(54) DEVICE FOR HAND REHABILITATION

(71) Applicant: De La Salle University, Manila (PH)

(72) Inventors: Nilo Bugtai, Manila (PH); Aira Patrice Ong, Manila (PH); Michael Manguerra, Manila (PH); Voltaire Dupo, Manila (PH); Maria Annyssa Perez, Manila (PH); Carlos Matthew Cases, Manila (PH); Eldrich Bong Valencerina, Manila (PH); Hanz Emmanuel Timbre, Manila (PH); Jeremy O. Flordelis, Cebu City (PH); Francis Carl Lau Belista, Manila (PH)

(73) Assignee: De La Salle University, Manila (PH)

(21) Appl. No.: 17/413,197

(22) PCT Filed: May 4, 2020
(86) PCT No.: PCT/PH2020/050005

§ 371 Date: Jun. 11, 2021

(30) Foreign Application Priority Data
May 6, 2019 (PH) 12019000184

(51) Int. Cl. A61H 1/02

(52) U.S. Cl. CPC A61H 1/0285; A61H 2201/165; A61H 2201/1671

(57) ABSTRACT

A device for hand rehabilitation includes a glove to be donned on a user's hand, and a wrist flexion assembly that facilitates passive or assisted ulnar/radial flexion of the user's wrist. The wrist flexion assembly includes a first wrist actuator and a second wrist actuator, each of which is attached to the glove. In one embodiment, a wrist rotation assembly is attached to the glove and cooperates with the wrist flexion assembly to provide for passive or assisted supination/pronation of the user's wrist. Further, at least one finger actuation assembly can be attached to the glove for passive or assisted flexion/extension of a corresponding finger of the user.

### FIELD OF THE INVENTION

The present invention relates generally to a device for physical rehabilitation, and particularly to a device for hand rehabilitation.

### DESCRIPTION OF THE RELATED ART

Stroke and injury may lead to loss of mobility in some parts of the body. In the Philippines, they remain the two leading causes of disability. Impairments of the hand are particularly troublesome, as the hand is needed for many important daily activities. Recent technological advances in biomedical engineering have paved the way for the development of devices for hand rehabilitation. These devices assist users in performing repetitive exercises and strengthening activities, which can help them regain motor function thereby improving their quality of life. The complexity of the human hand, however, continues to pose a challenge for the development of devices that can effectively increase hand motor recovery.

Several patent documents teach devices for hand rehabilitation. US20130072829 teaches a hand rehabilitation device that uses flexible rods to provide passive and assisted active, concurrent and/or selective, bending/extension of the five fingers. A rehabilitation system taught in US20120109025 improves performance of coordinated movements of at least one limb of a user, wherein the coordinated movements include active, assisted, and/or resistive forearm pronation, forearm supination, hand grasping, hand releasing, and isolated finger movements. US20150290071 describes a hand rehabilitation device that includes a linear actuation system to provide forces for exercising flexion and extension of a user's fingers, and a rotational actuation system to provide forces for exercising pronation and supination of a user's wrist.

In the present invention, a device for hand rehabilitation provides a means for passive or assisted ulnar/radial flexion of a user's wrist. The device may further include a means for passive or assisted wrist supination/pronation and/or a means for flexion/extension of at least one of the user's fingers.

### SUMMARY OF THE INVENTION

According to this invention, a device for hand rehabilitation comprises a glove to be donned on a user's hand, and a wrist flexion assembly that provides a means for passive or assisted ulnar/radial flexion of the user's wrist. The wrist flexion assembly includes a first wrist actuator and a second wrist actuator. Each of the first and second wrist actuators is attached to the glove.

In one embodiment, each of the first wrist actuator and the second wrist actuator includes a metacarpal cable positioning member, a wrist flexion cable attached on one end to the metacarpal cable positioning member, a wrist cable positioning element attached to the glove at a position corresponding to the user's wrist, and a linear wrist flexion motor. The metacarpal cable positioning member of the first wrist actuator is attached to the glove at a position corresponding to the dorsal side of the metacarpal of the index finger. The metacarpal cable positioning member of the second wrist actuator is attached to the glove at a position corresponding to the dorsal side of the metacarpal of the little finger. The wrist flexion cable passes through the wrist cable positioning element. The linear wrist flexion motor is connected to a portion of the wrist flexion cable opposite to the metacarpal cable positioning member and is operable to adjust the tension of the wrist flexion cable.

In one embodiment, the device for hand rehabilitation further comprises a support bar attached to the glove. The support bar links the metacarpal cable positioning member of the first wrist actuator and the metacarpal cable positioning member of the second wrist actuator.

In one embodiment, the device for hand rehabilitation further comprises a wrist rotation assembly attached to the glove. The wrist rotation assembly cooperates with the wrist flexion assembly to provide a means for passive or assisted supination/pronation of the user's wrist.

In one embodiment, the wrist rotation assembly includes a palmar cable positioning member, a wrist rotation cable attached on one end to the metacarpal cable positioning member, a wrist cable positioning member attached to the glove at a position corresponding to the user's wrist, and a linear wrist rotation motor. The palmar cable positioning member is attached to the glove at a position corresponding to the palmar side of the metacarpal of the little finger. The wrist rotation cable passes through the wrist cable positioning member. The linear wrist rotation motor is connected to a portion of the wrist rotation cable opposite to the palmar cable positioning member, and is operable to adjust the tension of the wrist rotation cable.

In one embodiment, the device for hand rehabilitation further comprises at least one finger actuation assembly attached to the glove for passive or assisted flexion/extension of a corresponding finger of the user.

In one embodiment, the finger actuation assembly includes a fingertip cable positioning member, a finger actuation cable attached on one end to the fingertip cable positioning member, a metacarpophalangeal cable positioning member, and a linear finger actuation motor. The fingertip cable positioning member is attached to the glove at a position corresponding to the distal interphalangeal joint of the corresponding finger at the dorsal side of the hand. The metacarpophalangeal cable positioning member is attached to the glove at a position corresponding to the metacarpophalangeal joint of the corresponding finger at the dorsal side of the hand. The finger actuation cable passes through the metacarpophalangeal cable positioning member. The linear finger actuation motor is connected to a portion of the finger actuation cable opposite to the fingertip cable positioning member, and is operable to adjust the tension of the finger actuation cable.

In one embodiment, the device for hand rehabilitation further comprises a spacer attached to the glove at a position corresponding to a region between the thumb and the index finger of the user's hand.

### BRIEF DESCRIPTION OF THE DRAWINGS

Other features and advantages of the invention will become apparent in the following detailed description of the embodiment with reference to the accompanying drawings, of which:

FIG. 1 is a perspective view of an embodiment of a device for hand rehabilitation according to this invention, the embodiment being worn on a user's right hand and viewed from the dorsal side of the right hand.

FIG. 2 is a perspective view of an embodiment of a device for hand rehabilitation according to this invention, the embodiment being worn on a user's right hand and viewed from the palmar side of the right hand.

FIGS. 3a and 3b are perspective views of an embodiment of a device for hand rehabilitation according to this invention, the embodiment being worn on a user's right hand and viewed from two different angles.

FIG. 4 is a block diagram showing linear wrist flexion motors of first and second wrist actuators, a linear wrist actuator motor of a wrist rotation assembly, and linear finger actuation motor of finger actuation assemblies of an embodiment of a device for hand rehabilitation according to this invention.

### DESCRIPTION OF THE EMBODIMENT

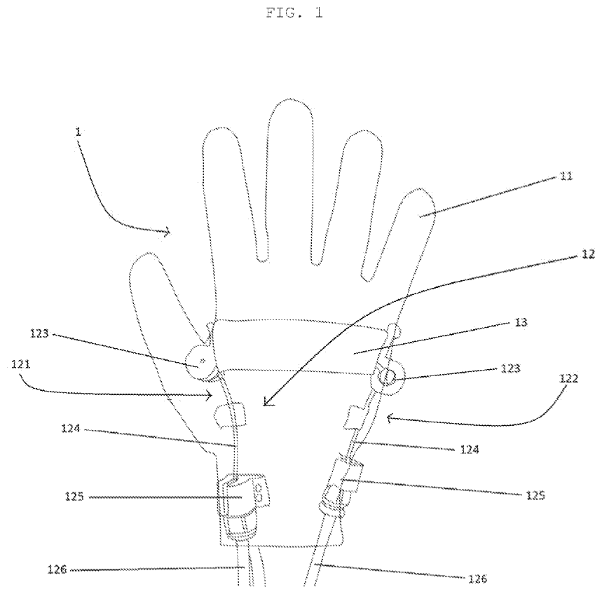

With reference to FIG. 1, a device (1) for hand rehabilitation according to an embodiment of this invention is shown to comprise a glove (11) to be donned on a user's hand and a wrist flexion assembly (12) providing a means for passive or assisted ulnar/radial flexion of the user's wrist. The wrist flexion assembly (12) includes a first wrist actuator (121) and a second wrist actuator (122). Each of the first wrist actuator (121) and the second wrist actuator (122) is attached to the glove (11).

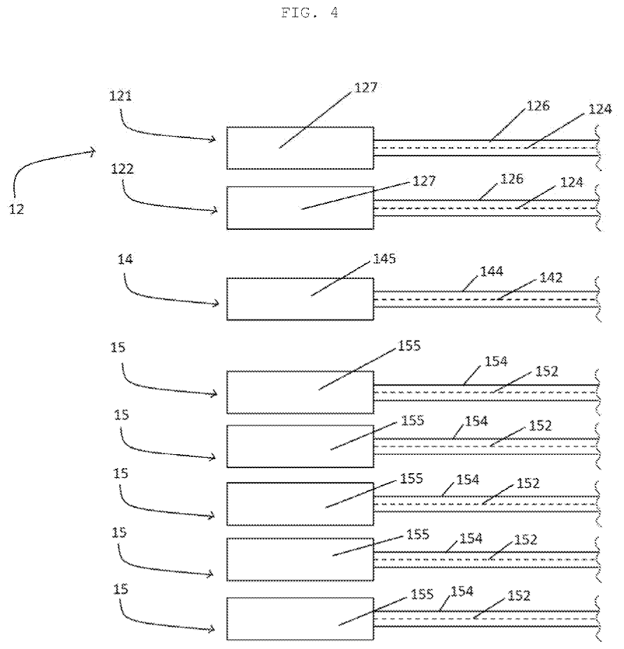

In the embodiment of the device (1) shown in FIGS. 1 and 4, the glove (11) is made of neoprene and cotton, but the invention is not limited in this respect. The glove (11) may include Velcro straps for adjusting the fit of the glove (11) on the user's hand. In addition, each of the first wrist actuator (121) and the second wrist actuator (122) includes: a metacarpal cable positioning member (123), a wrist flexion cable (124) attached on one end to the metacarpal cable positioning member (123), a wrist cable positioning element (125) attached to the glove (11) at a position corresponding to the user's wrist, and a linear wrist flexion motor (127) connected to a portion of the wrist flexion cable (124) opposite to the metacarpal cable positioning member (123). The metacarpal cable positioning member (123) of the first wrist actuator (121) is attached to the glove (11) at a position corresponding to the dorsal side of the metacarpal of the index finger. The metacarpal cable positioning member (123) of the second wrist actuator (122) is attached to the glove (11) at a position corresponding to the dorsal side of the metacarpal of the little finger. In this embodiment, the metacarpal cable positioning member (123) of the first and second wrist actuators (121, 122) are in the form of polylactic acid blocks and include holes to allow the metacarpal cable positioning member (123) to be sewn onto the glove (11). The metacarpal cable positioning member (123) may also be an acrylonitrile butadiene styrene (ABS) block. The wrist flexion cable (124) passes through the wrist cable positioning element (125) which, like the metacarpal cable positioning member (123) is in the form of a polylactic acid block and includes holes to allow the wrist cable positioning element (125) to be sewn onto the glove (11). The wrist cable positioning element (125) may also be a steel block. The wrist cable positioning element (125) may include a tube part (126) that extends to the linear wrist flexion motor (127). In one embodiment, the linear wrist flexion motor (127) is an L16 Acutronix 100 mm length linear motor with position indicator. The linear wrist flexion motor (127) is operable to adjust the tension of the wrist flexion cable (124). The tension of the wrist flexion cable (124) can be adjusted in a manner that causes the wrist flexion cable (124) to exert a push or a pull on the metacarpal cable positioning member (123) and consequently on the part of the user's hand corresponding to the location of the metacarpal cable positioning member (123) on the glove (11). The tube part (126) of the wrist cable positioning element (125) limits bending of the wrist flexion cable (124) when tension is adjusted.

To achieve an ulnar flexion, the first wrist actuator (121) is made to exert a push motion, while the second wrist actuator (122) is simultaneously made to exert a pull motion. To achieve a radial flexion, the first wrist actuator (121) is made to exert a pull motion, while the second wrist actuator (122) is simultaneously made to exert a push motion.

The embodiment of the device (1) in FIG. 1 also includes a support bar (13) that is attached to the glove (11) and that further assists in providing ulnar/radial flexion. The support bar (13), which may be made of PLA or ABS, links the metacarpal cable positioning member (123) of the first wrist actuator (121) and the metacarpal cable positioning member (123) of the second wrist actuator (122). In one embodiment, the support bar (13) is sewn onto the glove (11). The support bar (13) may also be further secured to the glove (11) using adhesives. In one embodiment, the metacarpal cable positioning member (123) of the first wrist actuator (121) is fused to one end of the support bar (13) and the metacarpal cable positioning member (123) of the second wrist actuator (122) is fused to the other end of the support bar (13).

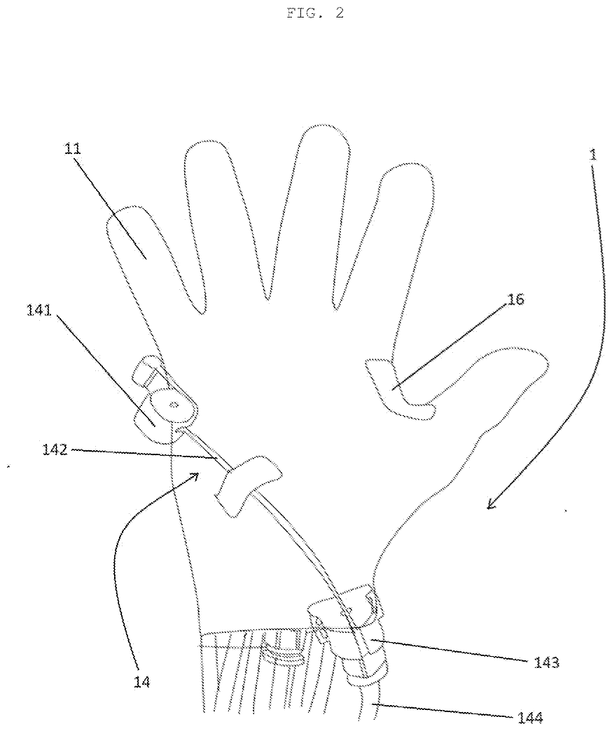

With reference to FIGS. 1 and 2, an embodiment of the device (1) further includes a wrist rotation assembly (14) attached to the glove (11). The wrist rotation assembly (14) cooperates with the wrist flexion assembly (12) to provide a means for passive or assisted supination/pronation of the user's wrist.

In the embodiment of the device (1) shown in FIGS. 2 and 4, the wrist rotation assembly (14) includes a palmar cable positioning member (141), a wrist rotation cable (142) attached on one end to the palmar cable positioning member (141), a wrist cable positioning member (143) attached to the glove (11) at a position corresponding to the user's wrist, and a linear wrist rotation motor (145) connected to a portion of the wrist rotation cable (142) opposite to the palmar cable positioning member (141). The palmar cable positioning member (141) is attached to the glove (11) at a position corresponding to the palmar side of the metacarpal of the little finger. The wrist rotation cable (142) passes through the wrist cable positioning member (143). In this embodiment, the palmar cable positioning member (141) and the wrist cable positioning member (143) are in the form of polylactic acid blocks and include holes to allow the palmar cable positioning member (141) and the wrist cable positioning member (143) to be sewn onto the glove (11). The palmar cable positioning member (141) may also be an ABS block. The wrist cable positioning member (143) may also be a steel block. The wrist cable positioning member (143) may include a tube part (144) that extends to the linear wrist rotation motor (145). In one embodiment, the linear wrist rotation motor (145) is an L16 Acutronix 100 mm length linear motor with position indicator. The linear wrist rotation motor (145) is operable to adjust the tension of the wrist rotation cable (142). The tension of the wrist rotation cable (142) can be adjusted in a manner that causes the wrist rotation cable (142) to exert a push or pull on the palmar cable positioning member (141) and consequently on the part of the user's hand corresponding to the location of the palmar cable positioning member (141) on the glove (11). The tube part (144) of the wrist cable positioning member (143) limits bending of the wrist rotation cable (142) when tension is adjusted.

To achieve a wrist supination, the wrist rotation assembly (14) is made to exert a pull motion, while the wrist flexion assembly (12) is simultaneously made to perform a radial flexion. To achieve a wrist pronation, the wrist rotation assembly (14) is made to exert a push motion, while the wrist flexion assembly (12) is simultaneously made to perform an ulnar flexion.

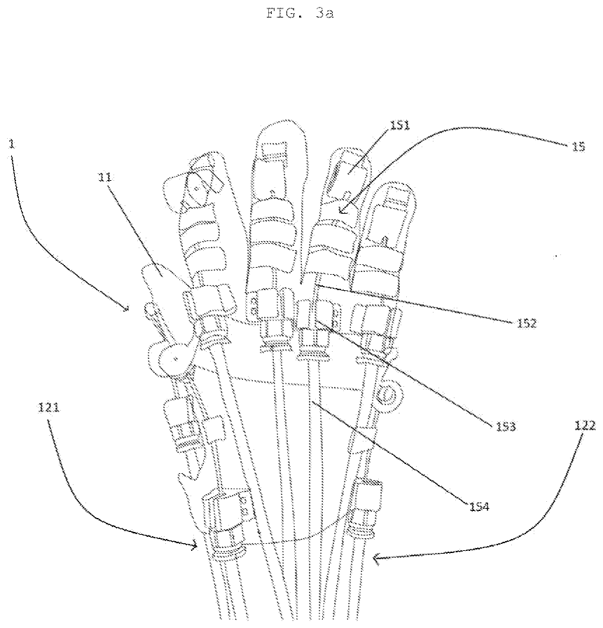

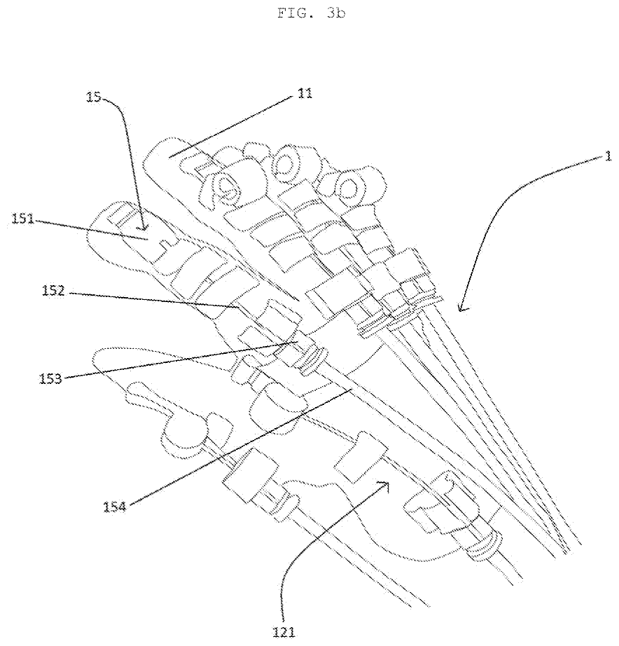

With reference to FIGS. 3a and 3b, an embodiment of the device (1) further includes at least one finger actuation assembly (15) attached to the glove (11) for passive or assisted flexion/extension of a corresponding finger of the user. The embodiment of the device (1) shown in FIGS. 3a and 3b includes finger actuation assemblies (15) for each of the five fingers of a user's hand.

In the embodiment of the device (1) in FIGS. 3a, 3b, and 4, each finger actuation assembly (15) includes a fingertip cable positioning member (151), a finger actuation cable (152) attached on one end to the fingertip cable positioning member (151), a metacarpophalangeal cable positioning member (153) attached to the glove (11) at a position corresponding to the metacarpophalangeal joint of the corresponding finger at the dorsal side of the hand, and a linear finger actuation motor (155) connected to a portion of the finger actuation cable (152) opposite to the fingertip cable positioning member (151). The fingertip cable positioning member (151) is attached to the glove (11) at a position corresponding to the distal interphalangeal joint of the corresponding finger at the dorsal side of the hand. The finger actuation cable (152) passes through the metacarpophalangeal cable positioning member (153). In this embodiment, the fingertip cable positioning member (151) and the metacarpophalangeal cable positioning member (153) are in the form of polylactic acid blocks and include holes that allow the fingertip cable positioning member (151) and the metacarpophalangeal cable positioning member (153) to be sewn onto the glove (11). The fingertip cable positioning member (151) may also be a ABS block. The metacarpophalangeal cable positioning member (153) may also be a steel block. The metacarpophalangeal cable positioning member (153) may include a tube part (154) that extends to the linear finger actuation motor (155). In one embodiment, the linear finger actuation motor (155) is an L16 Acutronix 100 mm length linear motor with position indicator. The linear finger actuation motor (155) is operable to adjust the tension of the finger actuation cable (152). The tension of the finger actuation cable (152) can be adjusted in a manner that causes the finger actuation cable (152) to exert a push or pull on the fingertip cable positioning member (151) and consequently on the part of the user's hand corresponding to the location of the fingertip cable positioning member (151) on the glove (11). The tube part (154) of the metacarpophalangeal cable positioning member (153) limits bending of the finger actuation cable (152) when tension is adjusted.

To achieve a finger flexion, the finger actuation assembly (15) is made to exert a push motion. In an embodiment of the device (1) having a finger actuation assembly (15), such as that shown in FIGS. 3a and 3b, the simultaneous finger flexion of all five fingers would cause a user to perform a hand closing or hand grasping motion. To achieve a finger extension, the finger actuation assembly (15) is made to exert a pull motion. Simultaneous finger extension of all five fingers would cause a user to perform a hand opening motion.

With reference to FIG. 2, an embodiment of the device (1) further includes a spacer (16) attached to the glove (11) at a position corresponding to a region between the thumb and the index finger of the user's hand. In this embodiment, the spacer (16) allows a user to form the “a-ok” sign with the thumb and index finger, when both fingers are simultaneously made to perform finger flexion. The spacer (16) may be made of PLA or ABS, and may have a structure similar to the LP 763 Wrist/Thumb Support. In one embodiment, the spacer (16) is sewn onto the glove (11) at six points on the glove (11): two points at positions corresponding to the base of the index finger, two points at positions corresponding to the base of the thumb, and two points at positions corresponding to the top of the space between the thumb and the index finger.

1. A device for hand rehabilitation, comprising:
a glove to be worn on a user's hand;
a wrist flexion assembly providing a means for passive or assisted ulnar/radial flexion of the user's wrist, the wrist flexion assembly including a first wrist actuator and a second wrist actuator, each of the first wrist actuator and the second wrist actuator being attached to the glove;
at least one finger actuation assembly attached to the dorsal outer side of the glove for passive or assisted flexion/extension of a corresponding finger of the user; and
a wrist rotation assembly attached to the glove, the wrist rotation assembly cooperating with the wrist flexion assembly to provide a means for passive or assisted supination/pronation of the user's wrist.

2. The device according to claim 1, wherein each of the first wrist actuator and the second wrist actuator includes:
a metacarpal cable positioning member, the metacarpal cable positioning member of the first wrist actuator being attached to the glove at a position corresponding to the dorsal side of the metacarpal of the index finger, the metacarpal cable positioning member of the second wrist actuator being attached to the glove at a position corresponding to the dorsal side of the metacarpal of the little finger;
a wrist flexion cable attached on one end to the metacarpal cable positioning member;
a wrist cable positioning element attached to the glove at a position corresponding to the user's wrist, the wrist flexion cable passing through the wrist cable positioning element; and
a linear wrist flexion motor connected to a portion of the wrist flexion cable opposite to the metacarpal cable positioning member, the linear wrist flexion motor being operable to adjust the tension of the wrist flexion cable.

3. The device according to claim 2, further comprising a support bar attached to the glove and linking the metacarpal cable positioning member of the first wrist actuator and the metacarpal cable positioning member of the second wrist actuator.

4. The device according to claim 1, wherein the wrist rotation assembly includes:
a palmar cable positioning member attached to the glove at a position corresponding to the palmar side of the metacarpal of the little finger;
a wrist rotation cable attached on one end to the palmar cable positioning member;
a wrist cable positioning member attached to the glove at a position corresponding to the user's wrist, the wrist rotation cable passing through the wrist cable positioning member; and
a linear wrist rotation motor connected to a portion of the wrist rotation cable opposite to the palmar cable positioning member, the linear wrist rotation motor being operable to adjust the tension of the wrist rotation cable.

5. The device according to claim 1, wherein the finger actuation assembly further includes:
a fingertip cable positioning member attached to the glove at a position corresponding to the distal interphalangeal joint of the corresponding finger at the dorsal side of the hand;
a finger actuation cable attached on one end to the fingertip cable positioning member;
a metacarpophalangeal cable positioning member attached to the glove at a position corresponding to the metacarpophalangeal joint of the corresponding finger at the dorsal side of the hand, the finger actuation cable passing through the metacarpophalangeal cable positioning member; and
a linear finger actuation motor connected to a portion of the finger actuation cable opposite to the fingertip cable positioning member, the linear finger actuation motor being operable to adjust the tension of the finger actuation cable.

6. The device according to claim 1, further comprising a spacer attached to the glove at a position corresponding to a region between the thumb and the index finger of the user's hand.
